(19) United States
(12) Patent Application Publication
(10) Pub. No.: US 2021/0358623 A1
(43) Pub. Date: Nov. 18, 2021

(54) A Method for Determining a DHEA Intensity Dose for Specific Patient

(71) Applicant: Eitan Nathan Eckstein, Petah Tikva (IL)

(72) Inventor: Eitan Nathan Eckstein, Petah Tikva (IL)

(21) Appl. No.: 16/319,334

(22) PCT Filed: Jul. 20, 2017
(86) PCT No.: PCT/IL2017/050818

§ 371 Date: Jan. 19, 2019

(51) Int. Cl. G16H 50/20; G16H 10/40; G16H 10/60; G16H 10/20; G16H 20/10

(52) U.S. Cl. CPC G16H 50/20; G16H 10/40; G16H 20/70; G16H 10/20; G16H 20/10; G16H 10/60

(57) ABSTRACT

A method operating on a processor or a machine learning processor for determining a specific dose of DHEA for a specific patient, by providing a dynamic predictive analytics using the biochemical parameters of the specific patient based on his blood test or saliva test, his molecular epigenetic map, his medical parameters regarding the level of his depression, anxiety, quality of life and intense of craving, and his psychological parameters.

### TECHNICAL FIELD

The present invention refers to a method for determining a DHEA intensity (dose per interval) for a specific patient based on his biochemical, molecular, epigenetic, medical, psychological, and his brain imaging parameters.

### BACKGROUND ART

Drug rehab includes a detoxification and rehabilitation periods. It is known that high percent of drug addicts who successfully went through the detoxification period failed in the rehabilitation period and unfortunately come back to drugs relapse Research has shown deficiencies in DHEA secretion in drug addicts. The DHEA is produced in the central nervous system and in the gonads and overy) during stressful situations the ratio of DHEA/cortisol is altered and this parameter is believed to be associated with the ability to cope with stress maladaptation. These raised the possibility that integration of exogenous DHEA application addiction treatment can enhance the rehabilitation process. Since the 90^{th} the DHEA is manufactured and marketed as a neurosteroid nutritional supplement.

In 2015, the inventors of the invention subject matter of the present patent application published the article: “Effect of dehydroepiandrosterone add-on therapy on mood, decision making and subsequent relapse of polydrug users”. In this article the inventors show that a DHEA treatment causes a significant decrease in detoxification symptoms, including depression and anxiety and also alleviate the intense craving for drugs in the rehabilitation period and enhance the chances for longer term success, decreasing relapse rate to drug usage.

In addition, the addicts cannot assimilate and implement new experiences, and remain affixed to their old experiences, which heighten drug craving. As their ability to acquire new habits is attenuated, and they cannot free themselves from the memories of cue-associated sensations of the drug offered Hence can (easily relapse) to their previous harmful drug-associated habits, even long time after conclusion of the rehabilitation program. The studies of the inventors demonstrated that DHEA treatment positively affected decision-making, mood and well-being as early as one month treatment, and had a long-lasting preventive effect on relapse to drug use. The DHEA-treated group reported fewer negative emotions, and showed more advantageous choosing in a decision-making task as compared to the placebo-treated patients in parallel to their cling period.

The inventors take their results a further step and developed a computer program to analyze the rehab process. They found out that the DHEA treatment is highly efficient when a specific patient receive a dose of DHEA which is tailored to his specific behavioral, cognitive and medical parameters. The inventors developed a method which employed algorithms, using mathematical and statistical calculations of the biochemical parameters, molecular epigenetic map, medical parameters, and psychological parameters of a specific patient for determining the intensity of DHEA treatment for a specific patient. The computer program also predicts the success of treatment and by that provides necessary information as to the period in which the patient should be supervised.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 describes schematically the main concept of the method that includes a computer processor (1), the biochemical parameters (BP), the molecular epigenetic map (MEM), the medical parameters (MP), the psychological parameters (PP), and the brain imaging (BI).

### THE INVENTION

The objective of the present invention is to provide a computer program for determining an intensity dose of DHEA for a specific patient. The method may be operated on a processor or on a machine learning processor, and uses the biochemical parameters (BP), the molecular epigenetic map (MEM), the medical parameters (MP), the psychological parameters (PP), and the brain imaging (BI) of the specific patient.

The biochemical parameters (BP) of the specific patient may be based on his blood test or saliva. These biochemical parameters include the level of DHEA, DHEA-S and Cortisol. The molecular epigenetic map (MEM) of the specific patient may be based on his blood test or saliva test. The medical parameters (MP) of the specific patient may be based on medical diagnosis regarding his level of depression, anxiety, and quality of life and intense of craving. These medical parameters may be based on the Hamilton rating scale for depression, Quality of Life Enjoyment and Satisfaction Questionnaires, Beck Anxiety Inventory, Brief Substance Craving Scale, PANAS scale and other kind of such questionnaires. The psychological parameters (PP) of the specific patient may be based on psychological questionnaire regarding his impulsiveness. These psychological parameters may be also based on the ASI Questionnaire, Iowa Gambling Task, and other kind of such questionnaires.

These data may be entered to the computer machine learning processor and after that the method includes the stage of assigning a particular weight to each of these biochemical parameters, molecular epigenetic map, each of the medical parameters, each of the psychological parameters, and the brain imaging. After this stage, the method can calculate dynamically/longitudinally the stage of estimating a weigh composition of these particulars cluster. This weigh composition enables to determine the optimized intensity dose of DHEA treatment for a specific addict longitudinally through the detoxification and rehabilitation progress. The method may further uses the brain imaging data of the specific patient, assigning a particular weight for this brain imaging for calculating and estimating the weigh composition.

The method can be used by rehab clinics which may enter these secure/confidential data to the computer program and receive the optimized intensity dose of DHEA for this specific patient as to its response to the treatment (machine learning). It is recommended to analyze data collected at the beginning of the detoxification period and then to repeat this action every month for several months consecutively. The method takes in account also the results of former data of the specific patient that were entered to the computer.

This weigh composition (predictive analytics) also enables to predict the probability of the specific patient to succeed in his rehabilitation process and therefore enables the rehab clinic to decide whether to release him of supervised care.

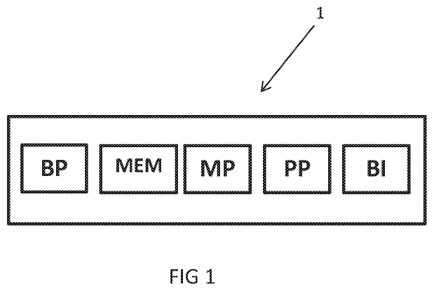

FIG. 1 describes schematically the main concept of the method that includes a computer (machine learning) processor (Processor), the biochemical parameters (BP), the molecular epigenetic map (MEM), the medical parameters (MP), the psychological parameters (PP), and the brain imaging (BI).

What is claimed is:

1. A method operating on a processor or a machine learning processor for determining an intensity dose of DHEA for a personalized medicine, comprising:
(a) providing biochemical parameters of said specific patient based on his blood test or saliva test wherein said biochemical parameters includes a level of DHEA and Cortisol;
(b) providing molecular epigenetic map of said specific patient based on his blood test or saliva test;
(c) providing medical parameters of said specific patient based on medical diagnosis regarding his level of depression, anxiety, quality of life and intense of craving;
(d) providing psychological parameters of said specific patient based on psychological questionnaire regarding his impulsiveness;
(e) providing said processor and entering to it said biochemical parameters, said molecular epigenetic map, said medical parameters, and said psychological parameters and assigning a particular weight to each of said biochemical parameters, said molecular epigenetic map, each of said medical parameters, and each of said psychological parameters;
(f) estimating a weigh composition of said particulars weights;
whereby said weigh composition enables to determine an optimized intensity dose of DHEA for said specific patient when he is addict during a detoxification and rehabilitation periods.

2. The method operating on a processor for determining an intensity dose of DHEA for a specific patient of claim 1, further comprising:
(a) providing a brain imaging data of said specific patient and entering said brain imaging data to said processor;
(b) assigning a particular predictive analytics weight for said brain imaging for calculating said estimation of said weigh composition.

3. A method for determining dose of DHEA for a specific patient, comprising:
(a) providing biochemical parameters of said specific patient based on his blood test or saliva test wherein said biochemical parameters includes a level of DHEA and Cortisol;
(b) providing molecular epigenetic map of said specific patient based on his blood test or saliva test;
(c) providing medical parameters of said specific patient based on medical diagnosis regarding his level of depression, anxiety, quality of life and intense of craving; side effects
(d) providing psychological parameters of said specific patient based on psychological questionnaire regarding his impulsiveness;
(e) assigning a particular weight to each of said biochemical parameters, said molecular epigenetic map, each of said medical parameters, and each of said psychological parameters; based on a specific algorithm
(f) estimating a weigh composition of said particulars weights;
whereby said weigh composition enables to determine an optimized the dose of DHEA for said specific addict during a detoxification and rehabilitation periods.

4. The method for determining an intensity of DHEA treatment for a specific patient of claim 3, further comprising:
(a) providing a brain imaging data of said specific patient;
(b) assigning a particular predictive analytics weight for said brain imaging for calculating said estimation of said weigh composition.
